(19) United States
(12) Patent Application Publication
(10) Pub. No.: US 2009/0123537 A1
(43) Pub. Date: May 14, 2009

(54) ORAL DOSAGE FORM OF CEFTRIAXONE AND METHODS OF USE

(76) Inventor: Daniel R. DeBrouse, Wellston, OK (US)

Correspondence Address: DUNLAP CODDING, P.C., PO BOX 16370, OKLAHOMA CITY, OK, 73113, US

(21) Appl. No.: 11/938,638

(22) Filed: Nov. 12, 2007

(51) Int. Cl. A61K 9/48; A61K 31/431

(52) U.S. Cl. 424/463; 514/196

(57) ABSTRACT

The present invention contemplates a novel oral dosage form for the intestinal delivery of ceftriaxone sodium. The oral dosage form inhibits enteric degradation of the therapeutic compound by encapsulation within an inner core region and having an outer shell, preventing its dissolution until reaching the small intestine. Furthermore, the enzymatic degradation of the compound is substantially inhibited until absorption at the intestinal mucosa.

### CROSS-REFERENCE TO RELATED APPLICATIONS

Not applicable.

### STATEMENT REGARDING FEDERALLY SPONSORED RESEARCH OR DEVELOPMENT

Not applicable.

### BACKGROUND OF THE INVENTION

The present invention relates generally to pharmaceutical and nutriceutical products, and, and more particularly to an improved method and apparatus for encapsulating pharmaceutical and nutriceutical bioactives, particularly ceftriaxone sodium, for intestinal delivery.

Ceftriaxone sodium is a broad spectrum third-generation cephalosporin antibiotic and is sold commercially as Rocephin® by Roche. Since the U.S. Food and Drug Administration first approved this antibiotic in 1985, physicians have successfully treated more than 100 million patients worldwide. Ceftriaxone sodium has been indicated for the eradication of bacterial pathogens involved for example in lower respiratory tract infections, skin and skin structure infections, urinary tract infections, uncomplicated gonorrheal, pelvic inflammatory diseases, bacterial septicemia, bone and joint infections, intra-abdominal infections, meningitis, surgical prophylaxis, as well as acute bacterial otitis media. The antibiotic has been used successfully in both adult and pediatric patients with minimal side effects. Unfortunately, however, conventional molecular science has yet to provide an effective oral delivery system for the administration of ceftriaxone to patients. Currently, effective bacteriocidal therapy using ceftriaxone hinges upon either intravenous infusion or intramuscular administration. Oral ceftriaxone formulations could beneficially reduce conventional burdens upon both patients and health care professionals as well, in addition to perhaps achieving higher blood ceftriaxone levels with lower dosages. However, oral ceftriaxone administration is entirely dependent upon the ability of the dosage form to deliver permeable solubilized antibiotic directly to the intestinal mucosa, rather than premature disintegration in gastric fluid.

The ceftriaxone sodium molecule presents as a white to yellowish-orange crystalline powder at 25° C., having the following chemical formula, C₁₈H₁₆N₈Na₂O₇S₃.3.5H₂O. Ceftriaxone sodium is (6R,7R)-7-[2-(2-Amino-4-thiazolyl)glyoxylamido]-8-oxo-3-[[(1,2,5,6-tetrahydro-2-methyl-5,6-dioxo-as-triazin-3-yl)thio]methyl]-5-thia-1-azabicyclo[4.2.0]oct-2-ene-2-carboxylic acid, 7²-(Z)-(O-methyloxime), disodium salt, sesquaterhydrate. Ceftriaxone is readily soluble in water, only sparingly to slightly soluble in methanol and ethanol respectively, and is relatively impermeable to the lipid fraction of cellular membranes. Ceftriaxone's relative impermeability is a result of its ionization and its degree of electrical resistance. The bactericidal activity of ceftriaxone results from inhibition of cell wall synthesis. Ceftriaxone has a high degree of stability in the presence of beta-lactamases, both penicillinases and cephalosporinases, of gram-negative and gram-positive bacteria.

Traditional ceftriaxone therapy is entirely dependent upon either intravenous (IV) infusion or intramuscular (IM) administration. Without wishing to be bound by theory, apparently there are at least three molecular characteristics contributing to ceftriaxone's limited therapeutic administration practices. First, the molecule displays a high degree of polarity. Although many polar molecules are know to transverse the mucosal lipid bilayer by passive transport through specialized pores, ceftriaxone absorption by this mechanism is remarkably poor. Second, ceftriaxone sodium is an ionized molecule, and as such is resistant to lipid dissolution, thus establishing an immediate barrier to the necessary lipid diffusion of ceftriaxone across the mucosal bilayer. Protein gated ion channels are well known components of the lipid bilayer, however, passage of molecules through such channels is usually restricted to inorganic ions much smaller than the ceftriaxone molecule. Thirdly, the ceftriaxone molecule presents a high degree of electrical resistance to the lipid bilayer due to its level of polarity. However, if these three barriers to lipid bilayer translocation alone were enough to prevent diffusion, many biologically active therapeutic agents incurrent use would have absolutely zero therapeutic value. It is clear, however, that drug absorption is also mediated by its concentration gradient, and in the case of charged molecules, the electrical potential difference across the cellular membrane. For example, Table 1 shows the average ceftriaxone plasma concentrations after single dose using IV administration, and Table 2 shows average plasma concentration after a single dose using IM administration. Plasma concentration drops substantially over time, and would result in a concommitent decrease in the concentration gradient near the mucosal wall.

TABLE 1
Plasma concentrations (ug/mL) of ceftriaxone
single dose IV administration.

| Dose | 0.5 hr | 1 hr | 2 hr | 4 hr | 6 hr | 8 hr | 12 hr | 16 hr | 24 hr |
|---|---|---|---|---|---|---|---|---|---|
| 1.0 g | 151 | 111 | 88 | 67 | 53 | 43 | 28 | 18 | 9 |

TABLE 2
Plasma concentrations (ug/mL) of ceftriaxone
single dose IM administration

| Dose | 0.5 hr | 1 hr | 2 hr | 4 hr | 6 hr | 8 hr | 12 hr | 16 hr | 24 hr |
|---|---|---|---|---|---|---|---|---|---|
| 1.0 g | 40 | 68 | 76 | 68 | 56 | 44 | 29 | ND | ND |

ND = Not Detected

Furthermore, 33-67% of a ceftriaxone dose was found excreted in the urine as unchanged drug. The remainder of a ceftriaxone dose was found secreted in bile and ultimately found microbiologically inactive in the feces. Such data clearly suggest ceftriaxone's poor absorbability.

A method of maintaining the therapeutic efficacy of ceftriaxone while enabling the drug to be taken orally would be of great medicinal benefit.

### BRIEF SUMMARY OF THE INVENTION

The present invention contemplates a novel oral dosage form for the intestinal delivery of ceftriaxone sodium. The oral dosage form inhibits enteric degradation of the therapeutic compound by encapsulation within an inner core region and having an outer shell, preventing its dissolution until reaching the small intestine. Furthermore, the enzymatic degradation of the compound is substantially inhibited until absorption at the intestinal mucosa.

It is therefore an object of the present invention to provide a new and improved method for encapsulating pharmaceutical and nutriceutical compounds, particularly ceftriaxone for oral administration of the enterically protected compound.

### BRIEF DESCRIPTION OF THE DRAWINGS

FIG. 1 is a representation of a ceftriaxone molecule showing an amide linkage as a possible site of protease cleavage within the intestinal fluid.

FIG. 2 is a representation of ceftriaxone indicating “amino acid like” residues therein.

FIG. 3 is a scheme showing a sodium-dependent amino acid transporter, wherein transport of an amino acid is dependent upon initial binding of a sodium ion and occurs through a symport process.

FIG. 4 is a cross-sectional view of a dosage form capsule of the present invention showing an inner core and an outer shell.

FIG. 5 is a cross-sectional view of a time release capsule form of the present invention showing two inner layers of ceftriaxone, additional layers may be added for extended control release.

FIG. 6 is a representation of a ceftriaxone molecule having linoleic acid coupled thereto.

### DETAILED DESCRIPTION OF THE INVENTION

The small intestine is the ideal dissolution target for a dosage of ceftriaxone, presuming one is able to overcome its characteristics of impermeability through the intestinal wall.

Several aspects of digestion must be considered in order to have an oral delivery system of ceftriaxone necessary for intestinal absorption and distribution into the blood and lymph. Mechanical digestion will initiate upon the swallowing of an oral tablet. Swallowing may be considered to consist the typical three steps, movement of the tablet through the mouth into the pharynx, through the pharynx into the esophagus, and finally through the esophagus and into the stomach. In the stomach, an oral dosage form of ceftriaxone must retain both its physical and chemical characteristics remaining relatively unchanged until passage into the duodenum. If the oral tablet were to disintegrate prematurely, i.e., in the stomach, absorbability of unsolubilized ceftriaxone would be poor at best and well under that provided by IV or IM administration. Next, chemical digestion must proceed after entry into the jejunum region of the small intestine. A proposed disaccharide component of the oral tablet will be hydrolyzed by the intestinal enzymes such as sucrase, lactase, and maltase thereby releasing solubilized ceftriaxone. Finally, mechanical digestion will again play a significant natural role by inducing the churning of the intestinal contents and bringing “lipid-solubilized” ceftriaxone in contact with the surface of intestinal mucosa. Although there is little supporting evidence, it should be noted that partial cleavage of the ceftriaxone molecule is possible by enzymatic components of the intestinal fluid. Therefore another chemical mechanism involving enzymatic inhibition in the local intestinal region of tablet disintegration shall be addressed regarding ceftriaxone delivery across intestinal mucosa.

The jejunum region of the small intestine is the ideal region for disintegration of an oral dosage form of ceftriaxone and thus release a “lipid-solubilized” ceftriaxone for two primary reasons. First, the small intestine is specialized for the digestion and subsequent absorption of digestive end products. Second, the small intestine maintains a large surface area prone to absorption, greatly increasing the probability of drug diffusion by one or any combination of at least four mechanisms. Premature disintegration in the stomach exposes ceftriaxone to a potentially degradative environment thereby resulting in an inadequate absorption of the antibiotic for therapeutic value. Likewise tablet disintegration within the large intestine would result in the excretion of a majority of the dose as waste, as is the primary function of the large intestine.

Thus, as is apparent from the description herein, the oral dosage form of the present invention is resistant to gastric disintegration, but readily dissolvable in the small intestine. The oral dosage form contemplated herein comprises an encapsulating formula designed to deliver both water and lipid soluble drugs intact to the jejunum region of the small intestine. Once passing through the duodenum, the dosage form readily disintegrates upon contact with digestive enzymes in the small intestines thereby releasing its solubilized therapeutic agent.

The oral dosage form encapsulation formula contemplated herein comprises two regions of distinct function, an inner core and an outer layer (also referred to herein as the outer shell or outer membrane). The inner core comprises a lipid solubilized amount of the therapeutic agent e.g., ceftriaxone held in conjunction with specific targeting molecules that home in on active transport receptor proteins lining the surface of intestinal cells. Such targeting molecules promote the active transport of the drug across the lipid bilayer of the cells where it may be processed and absorbed into the blood and lymph. In addition to targeting molecules, the inner core preferably contains both protectant molecules, which function to establish a barrier between the drug and the digestive pool until core collision with the intestinal surface, and a lipid solubilizing base medium, functioning in distinct separation of the inner core from that of the intestinally digestible outer shell and in bilayer dissolution of the core components.

An understanding of the intestinal mechanisms vital to the proposed oral ceftriaxone tablet absorption is useful and thus will be discussed below. Upon administration, the biologically active agent (i.e., drug) must transverse several semi-permeable cellular membrane barriers before reaching the systemic circulation. Such membranes generally exhibit a high degree of selectivity to the passage of drug molecules being composed primarily of a phospholipid bilayer. The lipid network, in addition to providing stability to the membrane, also displays globular proteins of various size and composition involved in processes including nutrient absorption, cellular regulation, and the transport of molecules across the lipid bilayer. In general, drugs may traverse this membrane barrier by any one of or combination of the following mechanisms; passive diffusion, facilitated passive diffusion, active transport, and pinocytosis.

In passive diffusion, transport across the bilayer is dependent upon the concentration gradient of the solute. For example, drug molecules able to transverse the bilayer by this mechanism are rapidly removed by the systemic circulation due to a high concentration at the site of dissolution and an initially low blood concentration, setting up a large difference in gradient. The prerequisites to simple diffusion are the lipid solubility of a drug, its degree of ionization, molecular weight, and the area of the absorptive surface. One function of the inner core of the oral tablet contemplated herein is to provide a lipid-solubilized component optimizing its potential chances of transport by simple diffusion. For example, ceftriaxone weakly attracted to the core lipid base components may rapidly be dispersed at the membrane surface, establishing a contacted difference in the concentration gradient between the gastrointestinal fluid and the blood.

In facilitated passive diffusion, a carrier component (protein) reversibly combines with a drug molecule at the exterior cell membrane and rapidly transports it across the bilayer, releasing the drug at the interior surface. Carrier mediated diffusion of this type is characterized by selectivity and saturability. This mode of transport is relatively rare in conventional drug administration and is limited by both the molecular configuration of the drug and the availability of carriers. Without wishing to be bound by theory, carrier-mediated diffusion is likely the most significant mode of oral ceftriaxone absorption. For example, as mentioned previously, special targeting molecules are present within the inner core of the dosage form contemplated herein. These interact with a specific type of carrier protein abundantly distributed over the surface of the exterior membrane. Such a mono-targeting mechanism in itself would be considered sufficient to achieve ceftriaxone absorption and thus the appropriate blood concentrations for successful therapy. However, two types of targeting molecules are weakly conjuncted within the inner core. The second type carries ceftriaxone to the connective tissue between intestinal cells where it may then be transported in complex by an abundant carrier operating within this region.

Active transport also is characterized by selectivity and saturability. However, here energy expenditure is required and active transport is limited to primarily endogenous substances. As may have been suspected active transport may be considered as a component to the oral ceftriaxone absorption process.

In drug absorption, pinocytosis is presumed to play a very minimal role and occurs mainly by chance presence of a drug molecule in the right place at the right time. In this process the cell membrane invaginates enclosing fluid and/or digestive particles thereby forming a vesicle that later detaches and moves to the cell interior. Although with protein drugs pinocytosis may be exploited to play a major absorptive role, here it is of little to no significance to ceftriaxone absorption.

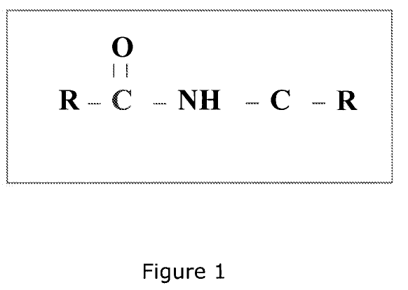

As mentioned earlier, ceftriaxone's limited degree of intestinal mucosal absorption is at least partially due to the polarity of the molecule, degree of ionization and its electrical resistance to the phospholipid bilayer. In addition to these, however, one further inhibitory process may be of critical concern. The amide linkage joining the traditional cephalosporin unit to one of the ceftriaxone specific side chains (see FIG. 1), closely resembles and thus possesses the characteristics of a peptide linkage as such, making the molecule susceptible to protease cleavage within the intestinal fluid and within the plasma as well, the latter of which may explain the administrative dose required for therapeutic value. Therefore, all strategic enhancement and translocation mechanisms must address such issues through protective measures upon tablet disintegration and subsequent ceftriaxone release.

In this section we shall discuss in some detail two separate prospective mechanisms of ceftriaxone translocation across the intestinal mucosa. Each individual mechanism corresponds to a likewise unique formulation and thus encapsulation aspect of the present invention.

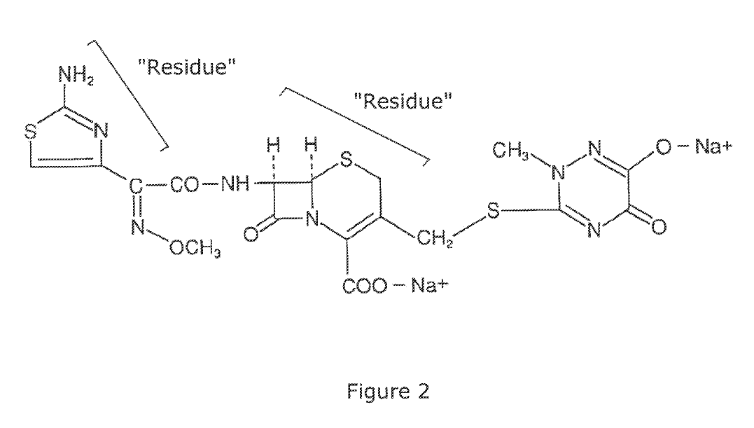

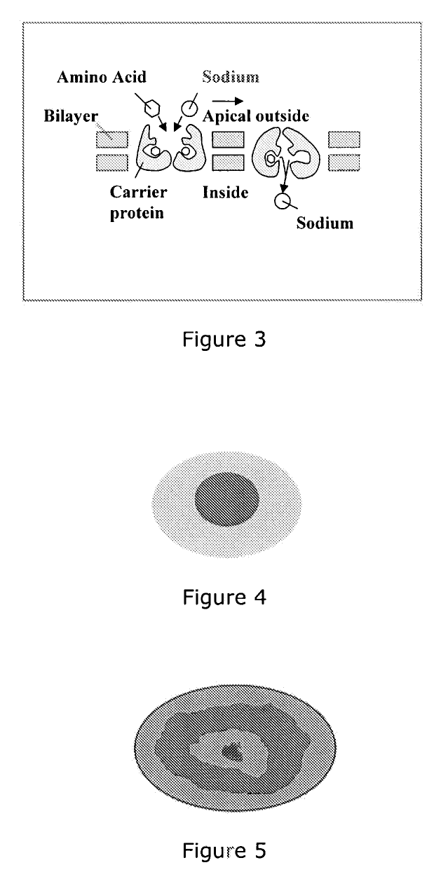

Examination of ceftriaxone's molecular structure reveals two moieties which resemble amino acids (FIG. 2). It is possible that provided optimal lumenal conditions, ceftriaxone may act like an amino acid or, di- or tri-peptide and be transported intracellularly by those transmembrane carriers involved in the uptake of such solutes. For example, at least four main types of sodium-dependent amino acid transporters are known to be present within the lumenal plasma membrane of the absorptive cells. These sodium-dependent transporters may bind amino acid solutes only after first binding a molecule of sodium. Without wishing to be bound by theory, this type of carrier transport process is thought to involve the transfer of solute across the lipid bilayer by undergoing reversible confirmational changes that alternately expose the solute binding sites first on one side of the membrane and then on the other. The interaction of solute with carrier resembles an enzyme-substrate reaction. Furthermore, the carrier may have one or more specific binding sites for its solute type that are exposed only after the initial binding of sodium ion by the carrier. FIG. 3 provides a schematic model of such a sodium-dependent amino acid transporter.

Without wishing to be bound by theory, ceftriaxone uptake, at least in part, may involve the active transport of the molecule by this type of sodium-dependent amino acid translocation process. It is clear that some dosage portion, although variable, of ceftriaxone is absorbed after oral administration of enterically coated capsules described herein (see Table 5 below). This strongly indicates the probability of such ceftriaxone translocation. Dose absorption is variable in part due to its dependence upon intestinal lumen sodium concentrations in the apical fluid surrounding the absorptive membrane. Intestinal fluid sodium concentrations would obviously vary from patient to patient depending upon dietary intake at, prior to and after dose administration.

In addition to the sodium-dependent translocation process described above, a similar calcium-dependent translocation mechanism may also be involved in ceftriaxone uptake at the apical membrane.

Experiments were conducted using several different ceftriaxone encapsulation dosage forms of the present invention for investigating this intestinal mucosal translocation process. Formula F1 dosage form involves the encapsulation of the ceftriaxone molecule within a delivery system specifically targeting intestinal lumen tablet disintegration. The F1 dosage form is absent any modulating and enhancing components. F1 simply delivers gastrically-protected ceftriaxone directly to the small intestine. Formula F2 dosage form also involves the intestinal delivery of protected ceftriaxone, with the addition of sodium chloride which is incorporated within the inner core of the dosage form as a translocation enhancement factor. Sodium chloride as an enhancer is added at a mole ratio sufficient to trigger sodium-dependent “amino acid” translocation of the ceftriaxone molecule. F2 is used to provide not only an increased bioavailability of ceftriaxone, but a more stable, less variable level of absorption of the drug at the mucosal membrane. Formula F3 also involves delivery of the drug to the apical surface of the intestinal mucosa and sodium chloride as an enhancer, as well as, additionally, a pH modulator which is intended to inhibit protease activity within the intestinal fluid immediately surrounding the region of tablet disintegration. As mentioned in earlier discussion, without wishing to be bound by theory, the so called “peptide” linkage present within the ceftriaxone molecule is believed to be susceptible to intestinal protease cleavage. If this is in fact the case, dose variability and bioavailability could also in-part be affected by protease activity. The pH modulator used herein (e.g., citric acid, or other acid having a similar effect) will be effective by lowering the pH of the intestinal fluid in the microenvironment immediately surrounding the ceftriaxone molecule and thus reversibly inhibits the enzymatic cleavage processes of the protease population. Results obtained using formulas F1, F2 and F3 and their corresponding tablet formulations (Table 3) and morphologies are discussed in detail below.

A second mechanism for ceftriaxone translocation is discussed below. The mechanisms of fat hydrolysis and subsequent component absorption across the apical membrane of an intestinal enterocyte are well known. For example, upon entry into the GI tract, fats are emulsified by bile salts. Next pancreatic lipase, which presents itself in large quantity upon meal consumption, cleaves the fat molecule at sites 1 and 3 of the triglyceride molecule liberating 2 free fatty acids and a molecule of 2-monoacylglycerol. The free fatty acid and 2-monoacylglycerol may then be absorbed from the intestinal lumen into polarized enterocytes lining the small intestine. Intensive studies regarding the uptake of free fatty acids and 2-monoacylglycerol across the brush-border membrane of enterocytes are available. In general, although simple diffusion of these molecules is known to occur on a limited scale, a considerable fraction of free fatty acid enters the enterocyte via a fatty acid transporter protein present on the apical brush-border membrane. Likewise, 2-monoacylglycerol is thought to interact with a long chain monoacylglycerol transporter through a related protein-mediated process for transport across the apical membrane. In fact there is evidence suggesting that transport of both molecules is possible by a single type of free fatty acid transport protein in a competitive manner. Following enterocyte absorption of free fatty acids and monoacylglyceride, these fat hydrolysis products are transported to the endoplasmic reticulum, where typically they are re-incorporated into triglyceride before shuttling through the golgi apparatus. Ultimately the newly re-incorporated triglyceride is packaged, through processing in these two organelles, with lipoproteins, cholesterol and other lipids into chylomicrons. Upon extrusion of the chylomicrons from the golgi apparatus an exocytotic vesicle is formed and transported to the basolateral aspect of the enterocyte. The vesicles then undergo exocytosis by fusing with the plasma membrane, dumping the chylomicrons into the extracellular matrix. Instead of being absorbed directly into capillary blood, the chylomicron particles are transported through the lymphatic system for disassembly and rapid delivery to the blood.

Two additional oral ceftriaxone dosage formulations, F4 and F5, may also be utilized herein for the enhancement of bioavailability of ceftriaxone. Formula F4 involves an enhanced “piggy-back” translocation using long-chain fatty acid transporters to carrier ceftriaxone across the bilayer by “loosely” conjuncting a molecule of a fatty acid, e.g., linoleic acid, to the ceftriaxone molecule. It should be noted that the association of these two molecules would be through attractive forces only and would not constitute their covalent bonding to one another. Formula F4 enhances the bioavailability of ceftriaxone through a “piggy-back” translocation.

In another formula, F5, a coupled translocation is involved wherein ceftriaxone is covalently linked directly to a molecule of a fatty acid via the amino terminus of the ceftriaxone molecule through a “peptide” linkage. In this way ceftriaxone is transported directly by the fatty acid molecule as it is transported by its corresponding enterocyte carrier.

The dosage formulations contemplated herein are suitable for the encapsulation and subsequent intestinal delivery of a broad spectrum of hydrophobic and hydrophilic biologically active, therapeutic molecules. Further, with minor formulation adjustments, the delivery of proteins, DNA and RNA molecules is possible as well. Both immediate and controlled release formulations have been developed for the delivery of bioactive molecules over a period ranging from 1-24 hours. Discussed below is a review of several examples of the dosage forms as contemplated within the scope of the present invention.

The dosage forms contemplated herein (referred to also as “Lipid Pherma” encapsulation) may be generated in various configurations of size, diameter, and shape. In a preferred embodiment, the dosage form is a spherical to oval shaped capsule or tablet. In its simplest form, the capsule consists of two separate regions or domains of distinct function, one is an inner core region, which is surrounded by a second layer, an outer protective membrane or shell (FIG. 4). The inner core functions in the release of both drug and absorbance enhancement components important to enterocyte uptake. The outer shell serves to protect the inner core from digestion in the gastric environment and to deliver the therapeutic compound intact to the intestinal mucosa.

As mentioned previously, the inner core region is theoretically un-limited in its potential bioactive delivery. All that is required is that the molecule has some preference to enterocyte uptake at the apical absorptive bilayer. This preference may be either natural or artificial in the sense of the addition of molecules proven to enhance the bioactives measured translocation across the apical membrane.

The inner core region of the present dosage form configuration contains an administrative dose of ceftriaxone sodium equivalent to 40-1000 mg of active antibiotic. In addition to the antimicrobial component, the inner core also may contain 0.00001-0.005 moles of translocation enhancer, such as sodium chloride or calcium chloride (and more preferably 0.00002-0.002 moles of NaCl), a mono-, di- or tri-saccharine, glycerin, fatty acid such as linoleic, or any other molecule of known ceftriaxone enhancement capabilities and combinations thereof for example as shown as formulas F2-F5 (Tables 3 and 4). The translocation enhancer may be added in higher mole ratios than shown therein depending upon the specific choices. The inner core region may house a pH modulating agent as well, as noted above. For example, an acid (e.g., citric acid) may be added as a pH modulator to lower the local intestinal luminal pH upon tablet disintegration and ceftriaxone release. Although any of a number of acids (such as fruit acids including lactic acid, malic acid, alpha hydroxy acids, glycolic acid, citric acid) could be used for this purpose, citric acid is preferred at a level of around 0.006 g/Kgbw. A pH modulator provides the benefit of reversible pH inhibition of intestinal luminal enzymatic activity thus mitigating ceftriaxone cleavage prior to absorption.

A protease inhibitor may be included in the inner core as well. Examples of such protease inhibitors include, but are not limited to, AEBSF-HCI, Amastatin-HCI, (epsilon)-Aminocaproic acid, (alpha)1-Antichymotrypsin from human plasma, Antipain-HCL, Antithrombin III from human plasma, (alpha)1-Antitrypsin from human plasma, (4-Amidinophenyl-methane sulfonyl-fluoride), Aprotinin, Arphamenine A, Arphamenine B, Benzamidine-HCI, Bestatin-HCI, CA-074, CA-074-Me, Calpain Inhibitor I, Calpain Inhibitor II, Cathepsin Inhibitor Z-Phe-Gly-NHO-Bz-pMe, Chymostatin, DFP (Diisopropylfluoro-phosphate), Dipeptidylpeptidase IV Inhibitor H-Glu-(NHO-Bz)Pyr, Diprotin A, E-64, E-64d (EST), Ebelactone A, Ebelactone B, EDTA-Na₂, EGTA, Elastatinal, Hirudin, Leuhistin, Leupeptin-hemisulfate, (alpha)2-Macroglobulin from human plasma, 4-(2-Aminoethyl)-benzenesulfonyl fluoride hydrochloride, Pepstatin A, Phebestin, Phenylmethyl sulfonyl fluoride, Phosphoramidon, (1-Chloro-3-tosylamido-7-amino-2-heptanone HCI, (1-Chloro-3-tosylamido-4-phenyl-2-butanone), Trypsin inhibitor from egg white (Ovomucoid), and Trypsin inhibitor from soybean.

Finally the inner core may contain a small amount of an emulsifier such as Tween 20, or others known in the art. Examples of emulsifiers which may be used include but are not limited to non-ionic, anionic, cationic and amphoteric surfactants such as are commercially available, for example from Sigma Aldrich Co. (www.sigmaaldrich.com). Specific examples include 2-Cyclohexylethyl β-D-maltoside, Brij® 30, Brij® 56, Brij® 72, Decyl β-D-maltopyranoside, Diethylene glycol monodecyl ether, Diethylene glycol monohexadecyl ether, Diethylene glycol monopentyl ether, Ethylene glycol monodecyl ether, Ethylene glycol monohexadecyl ether, Heptaethylene glycol monododecyl ether, N-Decanoyl-N-methylglucamine, N-Nonanoyl-N-methylglucamine, N-Octanoyl-β-D-glucosylamine, Nonyl β-D-glucopyranoside, Octaethylene glycol monodecyl ether, Octaethylene glycol monohexadecyl ether, Pentaethylene glycol monodecyl ether, Polyoxyethylene 10 tridecyl ether, Polyoxyethylene 20 oleyl ether, Polyoxyethylene 40 stearate, Pril® neutral detergent, Saponin, Span® 20, Span® 60, Span® 80, Sucrose monodecanoate, Sucrose monolaurate, TWEEN® 20, TWEEN® 40, TWEEN® 80, TWEEN® 85, Tergitol® NP-9, Tergitol® TMN 10, Tergitol®, Tergitol® Type 15-S-12, Tetraethylene glycol monodecyl ether, Tetraethylene glycol monohexadecyl ether, Tetraethylene glycol monooctadecyl ether, Tetramethylammonium hydroxide pentahydrate, Triethylene glycol monodecyl ether, Triethylene glycol monooctadecyl ether, Triton®, Triton® N-60, Triton® X-100, Triton® X-102, Triton® X-15, Triton® X-207, Triton® X-45, Triton® XL-80N, Tyloxapol, Undecyl-β-D-maltoside, n-Dodecyl β-D-maltoside, 1-Octanesulfonic acid sodium salt, Chenodeoxycholic acid, Cholic acid, Dehydrocholic acid, Deoxycholic acid, Glycocholic acid hydrate, Lithium dodecyl sulfate, N,N-Dimethyldodecylamine N-oxide solution, N-Lauroylsarcosine sodium, Niaproof™ 4, Sodium 1-butanesulfonate, Sodium 1-decanesulfonate, Sodium 1-dodecanesulfonate, Sodium 1-heptanesulfonate, Sodium 1-nonanesulfonate, Sodium 1-propanesulfonate, Sodium cholate, Sodium choleate, Sodium deoxycholate monohydrate, Sodium dodecyl sulfate, Sodium dodecylbenzenesulfonate, Sodium glycodeoxycholate, Sodium hexanesulfonate, Sodium octyl sulfate, Sodium pentanesulfonate, Sodium taurochenodeoxycholate, Triton® QS-15, Triton® QS-44, Triton® X-200, Triton® XQS-20, Trizma® dodecyl sulfate, Ursodeoxycholic acid, Alkyltrimethylammonium bromide, Amprolium hydrochloride, Benzalkonium chloride, Benzethonium chloride, Benzyldimethylhexadecylammonium, Choline p-toluenesulfonate, Denatonium benzoate, Dimethyldioctadecylammonium bromide, Ethylhexadecyldimethylammonium bromide, Hexadecylpyridinium bromide, Hexadecyltrimethylammonium bromide, Hyamine® 1622, Luviquat™ FC 370, Luviquat™ HM 552, Methylbenzethonium chloride, Tetraheptylammonium bromide, Tetrakis(decyl)ammonium bromide, 3-[N,N-Dimethyl(3-palmitoylaminopropyl)ammonio]-propanesulfonate, ASB-14, EMPIGEN® BB detergent, N-Dodecyl-N,N-dimethyl-3-ammonio-1-propanesulfonate, and Sodium 2,3-dimercaptopropanesulfonate monohydrate.

The inner core region as described above preferably is a liquid droplet with a typical viscosity greater than that of milk although the inner core may be solid or semi-solid as well.

The second layer, a protective outer shell over the inner core, also commonly referred to herein as the outer membrane, is made up of an alginate, preferably initially of sodium alginate, which is then converted into a gastricly insoluble calcium form (calcium alginate) in stage one of capsule (tablet) manufacture. In one embodiment, the calcium alginate is present in a mole ratio of 3:1 in comparison to that of ceftriaxone. Small amounts of high fructose corn syrup, 1 mole ceftriaxone bases, and glycerin optionally may be added to aide in polymerization and membrane stability. The outer shell preferably has a texture consistent with that of a rubbery material and is extremely resistant to gastric disintegration, but undergoes a dissolution process upon entering the small intestine. Because of these unique chemical properties the second layer may easily be formulated into a controlled release format for dose delivery up to a period of 24 hours. This may be accomplished through a consecutive layering of alginate polymer outer shells with inner core layers comprising ceftriaxone dosages, as shown in FIG. 5.

With the basic conceptual dosage form configuration and formulas now presented, the chemical properties of these capsules and the ceftriaxone encapsulation process will be examined. For convenience the three sodium-dependent transportation formulas (F1, F2, F3) and their properties shall be discussed separately from those of the long-chain fatty acid translocation processes (F4, F5).

As noted above, three test formulations (F1, F2, F3) were devised to evaluate the enhancement of ceftriaxone translocation via a sodium dependent mechanism. In discussion we will further define each of these three formulations separately with the following designations; F1=“naked encapsulation”, F2=Enhancer mediated “naked encapsulation”, and F3=Enhancer mediated assisted modulator “naked encapsulation.” The encapsulation process (formation of the outer shell of alginate) itself is sequentially identical for each of the individual formulations with the primary variance being in the ingredient compositions of the inner core region. Specific (non-limiting) embodiments of the formulations are shown in Table 3.

TABLE 3
Composition of Formulas F1, F2 and F3.

| | Outer Shell | |
|---|---|---|
| Formulation | mol/1 L | Inner Core |
| F1 | CaAlginate | Ceftriaxone |
| | 0.17 | 0.00002-0.002 mol |
| | Glycerin | |
| | 0.043 | |
| | Fructose 0.1 | Tween 20* |
| | Water 55.0 | |
| F2 | CaAlginate | Ceftriaxone |
| | 0.17 | 0.00002-0.002 mol |
| | Glycerin | Tween 20* |
| | 0.043 | |
| | Fructose 0.1 | NaCl |
| | | 0.00002-0.002 mol |
| | Water 55.0 | |
| F3 | CaAlginate | Ceftriaxone |
| | 0.17 | 0.00002-0.002 mol |
| | Glycerin | Tween 20* |
| | 0.043 | |
| | Fructose 0.1 | NaCl |
| | | 0.00002-0.002 mol |
| | Water 55.0 | Citric Acid |
| | | 0.031 mol/Kgbw |

*Tween 20 is used in minimal amount as emulsifier.

The outer shell of the ceftriaxone dosage form is both considered and prepared separately from the inner core region. It should be understood that the procedure described herein for the preparation of the described capsules is conducted under a research setting and it will be understood by a person of ordinary skill in the art that the dosage form may be manufactured on an industrial scale by any of a number of processes in current practice known to persons having ordinary skill in the art.

Encapsulation Procedure: Preparation of the Outer Membrane (Outer Shell)

An appropriate amount of a stock outer membrane (outer shell) solution is prepared to support the amount of dosage forms one wishes to generate. For example, the outer membrane of each capsule (dosage form) may comprise approximately 1.0 mL of outer membrane stock solution. Therefore if one wishes to prepare 10 capsules, at least 10 mL stock solution would be prepared. The stock solution is prepared using cold water for ease of homogeneity. Once thoroughly mixed the solution is heated at 100° C. for a period of 1-5 minutes. At the end of the heating period, 1.0 mL of hot stock membrane solution is quickly poured into a specialized capsule mold and immediately submerged into a calcium chloride solution. Upon submersion into aqueous calcium chloride, a sodium ion from the alginate molecule exchanges with a calcium ion from the added calcium chloride solution. The ionic change results in the generation of an alginate biopolymer resistant to gastric digestion (as explained above) which is then immediately impregnated with a solution comprising ceftriaxone to form the inner core. The inner core is prepared by simply emulsifying the therapeutic agent, translocation enhancer and pH modulator in an amount of Tween 20 and vortexed (e.g., for a period of 60 seconds) prior to microinjection into the lumen of the outer shell layer. Inner core microinjection should proceed immediately after the submersion of the outer shell into the calcium chloride solution, about 30-60 seconds. After impregnation, the capsules are re-submerged into calcium chloride solution and allowed to stand to reduce moisture in the outer shell, about 3-10 minutes. It should be understood that the capsules may be removed from calcium chloride solution as early as 10-20 seconds after the second submersion process, depending upon the desired percent moisture content. Once the initial capsule has been prepared according to the above process it may optionally be coated with an additional functional or cosmetic membrane, shell, or coating such as a carrageenan outer shell, and dried accordingly as desired.

As noted above, formulas F4 and F5 promote ceftriaxone bioavailability enhancement through fatty acid translocation. Formula F4, “non-traditional enhanced “piggy-back” translocation”, makes use of a molecule of linoleic acid as enhancer for translocation across the enterocyte membrane. Here the outer shell is prepared as above, differing only in its inner core component formulation.

TABLE 4
Compositions of formulas F4 and F5.

| Formulation | Outer Shell mol/1 L | Inner Core |
|---|---|---|
| F4 | CaAlginate 0.17 | Ceftriaxone |
| | Glycerin 0.043 | 0.00002-0.002 mol |
| | Fructose 0.1 | Linoleic Acid |
| | Water 55.0 | 0.2 mol/L |
| F5 | CaAlginate 0.17 | CeftriaxoneLinA |
| | Glycerin 0.043 | 0.00002-0.002 mol |
| | Fructose 0.1 | |

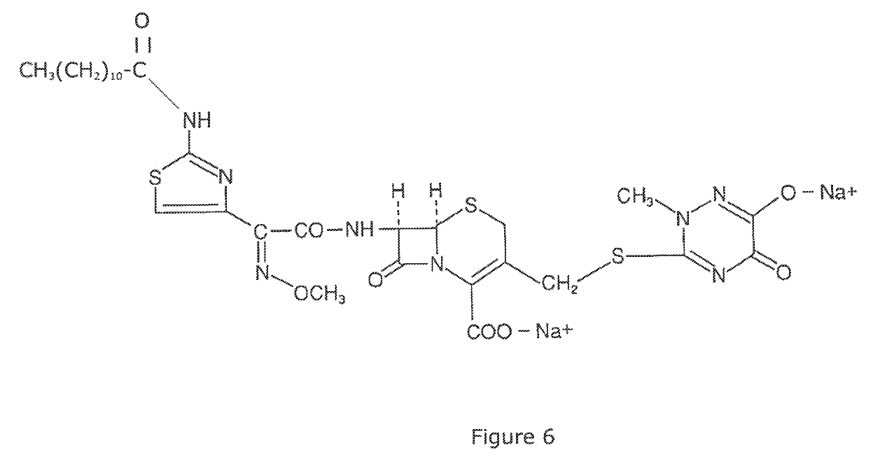

The outer shell of Formula F5, the “non-traditional coupled translocation” formulation, is again prepared in a manner identical to that outlined for above. Here, instead, a molecule of a fatty acid such as linoleic acid is covalently linked to the amino terminus of the ceftriaxone molecule (see FIG. 6) in a manner known to persons of ordinary skill in the art of organic synthesis. It should be understood that other types of fatty acid molecules could be used here with comparable enhancement, including but not limited to myristoleic acid, palmitoleic acid, oleic acid, linoleic acid, alpha-linolenic acid, arachidonic acid, eicosapentaenoic acid, erucic acid, and docosahexaenoic acid.

Experimental:

Oral ceftriaxone capsules as contemplated in accordance with the present invention have been administered to rabbits. New Zealand White Rabbits were obtained from Harlan Inc. Each rabbit was approximately 10 lbs. Rabbits were housed at the MidWest City Veterinary Hospital until the time of the study. Multiple rabbits were dosed with formulations F1-F4 and absorption data was collected by hplc analysis of plasma ceftriaxone concentrations. In general, blood draws were collected at pre-determined intervals of time and spun down to separate the serum, which was then removed and frozen prior to delivery to Clinical Pathology Laboratories in Oklahoma City, Okla. Current IACUC methods for oral administration and blood collection were followed. All rabbits were dosed at a ceftriaxone equivalent of 60 mg/dose (oral or IV) (a ceftriaxone load of 40-80 mg depending upon body weight). Each dose was for animals 001-005 administered intact by oral route through a specially designed capsule delivery tube by a reputable veterinarian or by IV for animal 006. Results are shown in Table 5.

TABLE 5

| | | | | | | | |
|---|---|---|---|---|---|---|---|
| Animal | | | | | | 4.0 | 6.0 |
| Number | Formula | 0.00 Hours | 0.5 Hours | 1.0 Hours | 2.0 Hours | Hours | Hours |

Plasma Ceftriaxone Concentrations (μg/mL)

| | | | | | | | |
|---|---|---|---|---|---|---|---|
| 001 | F1 | BLQ | 31.84 | 21.71 | 14.47 | 5.79 | 4.34 |
| 002 | F2 | BLQ | 110.88 | 75.60 | 50.40 | 20.16 | 15.12 |
| 003 | F3 | BLQ | 143.15 | 97.60 | 65.07 | 26.03 | 19.52 |
| 004 | F4 | BLQ | 35.69 | 24.33 | 16.22 | 6.44 | 4.87 |
| 005* | F2/F3 | BLQ | 40.194 | 27.41 | 18.27 | 7.31 | 5.48 |

Plasma Concentration of Ceftriaxone Over
Time After IV Administration of Ceftriaxone Sodium

| | | | | | | | |
|---|---|---|---|---|---|---|---|
| 006 | Control | BLQ | 198.21 | 135.63 | 90.81 | 36.43 | 27.34 |

BLQ = Below Limit of Quantitation (<1.00 μg/mL)
*Rabbit 005 was dosed with a combination of Formulas F2 and F3.

These data demonstrate that the enterically-coated oral dosage formulations of the present invention, including F1 and F2 and F3 which comprise NaCl, and formulations F4 and F5 which comprise fatty acid components in the inner core, provide ceftriaxone plasma concentrations which are sufficient to provide a therapeutic effect in mammals, including humans.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, compositions of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. An oral dosage form comprising:
an inner core comprising a suspension of a therapeutic compound, and an emulsifier; and
an enteric coating covering the inner core, wherein the enteric coating comprises calcium alginate, and wherein the oral dosage form is resistant to digestion in the stomach such that the dosage form is maintained substantially intact until it enters the small intestine wherein the therapeutic compound is released from the inner core.

2. The oral dosage form of claim 1 wherein the therapeutic compound is ceftriaxone sodium.

3. The oral dosage form of claim 1 wherein the inner core further comprises a membrane translocation enhancer.

4. The oral dosage form of claim 3 wherein the membrane translocation enhancer further comprises NaCl.

5. The oral dosage form of claim 1 wherein the inner core further comprises a pH modulating agent.

6. The oral dosage form of claim 5 wherein the pH modulating agent is an acid.

7. The oral dosage form of claim 6 wherein the acid is a fruit acid.

8. The oral dosage form of claim 7 wherein the fruit acid is citric acid.

9. The oral dosage form of claim 1 wherein the emulsifier is Tween 20.

10. The oral dosage form of claim 1 wherein the inner core further comprises a long chain fatty acid.

11. The oral dosage form of claim 10 wherein the long chain fatty acid is covalently-linked to the therapeutic compound.

12. The oral dosage form of claim 1 wherein the enteric coating further comprises glycerin and fructose.

13. An oral dosage form for enteric delivery, comprising:
an inner core comprising ceftriaxone sodium, NaCl, citric acid, and an emulsifier; and
an enteric coating comprising calcium alginate, and wherein the oral dosage form is resistant to digestion in the stomach such that the dosage form is maintained substantially intact until it enters the small intestine wherein the ceftriaxone sodium is released from the inner core.

14. A method of orally delivering ceftriaxone sodium to a patient in therapeutic need thereof, comprising:
providing an oral dosage form comprising an inner core comprising a suspension of ceftriaxone sodium, and an emulsifier, and an enteric coating covering the inner core, wherein the enteric coating comprises calcium alginate, and wherein the oral dosage form is resistant to digestion in the stomach such that the dosage form is maintained substantially intact until it enters the small intestine wherein the ceftriaxone sodium is released from the inner core; and
orally administering the oral dosage form to the patient wherein the ceftriaxone sodium is released into the small intestine.

15. The method of claim 14 wherein the inner core further comprises a membrane translocation enhancer.

16. The method of claim 14 wherein the inner core further comprises a pH modulating agent.

17. The method of claim 14 wherein the inner core further comprises a long chain fatty acid.

18. The method of claim 17 wherein the long chain fatty acid is covalently-linked to the ceftriaxone.

19. The method of claim 14 wherein the enteric coating further comprises glycerin and fructose.

20. A method of orally delivering ceftriaxone sodium to a patient in therapeutic need thereof, comprising:
providing an oral dosage form comprising an inner core comprising ceftriaxone sodium, NaCl, citric acid, and an emulsifier, and an enteric coating over the inner core, the enteric coating comprising calcium alginate, and wherein the oral dosage form is resistant to digestion in the stomach such that the dosage form is maintained substantially intact until it enters the small intestine wherein the ceftriaxone sodium is released from the inner core; and
orally administering the oral dosage form to the patient wherein the ceftriaxone sodium is released into the small intestine.
